(19) United States
(12) Patent Application Publication
(10) Pub. No.: US 2024/0041175 A1
(43) Pub. Date: Feb. 8, 2024

(54) Smartphone Combo Wallet Sleeve with Clip and Pop-Up Stand, with Magnetic Key Fob and Attachment Disk

(71) Applicant: Fahed Hishmeh, Nicholasville, KY (US)

(72) Inventor: Fahed Hishmeh, Nicholasville, KY (US)

(21) Appl. No.: 18/381,847

(22) Filed: Oct. 19, 2023

Related U.S. Application Data
(60) Provisional application No. 61/418,774, filed on Dec. 1, 2010.

(51) Int. Cl. A45C 11/32; A45C 11/18

(52) U.S. Cl. CPC A45C 11/321; A45C 11/182; A45C 2011/002

(57) ABSTRACT

A smartphone wallet sleeve combo with both a clip and a pop-up stand. The clip is attached at the bottom of the wallet sleeve so that when attached to clothing the opening of the wallet sleeve is oriented upward to keep cards from falling out of the wallet sleeve. The pop-up stand includes an attachment magnet. The invention also includes a magnetic key fob that includes a key ring and a disk end with attachment disks on both sides. The invention also includes an attachment disk with an adhesive side to attach to a flat surface and a magnetic side to attach to either the key fob or the wallet sleeve, or both.

### CROSS REFERENCE TO RELATED APPLICATIONS

This application claims priority to U.S. provisional application Ser. No. 61/418,774, filed on Oct. 24, 2022, and incorporated fully herein by reference.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

### Field of the Invention

The present invention relates to a smartphone wallet sleeve with an integrated clip and pop-up stand having an attachment magnet, and that works in conjunction with a magnetic key fob having a paired attachment magnet, and an attachment disk having adhesive on one side and an attachment magnet on the other side. Each component includes an attachment magnet that allows each to attach to the other. The attachment disk can be mounted in a convenient place. This allows the key fob to attach to the wallet, the key fob attach to the fixed attachment disk, the phone to attach to the key fob or attachment disk, and the phone to attach to the keys, and the attachment disk to secure the phone, wallet, and keys in a convenient place.

### Description of the Related Art

There are numerous smart phone wallet sleeves, pop-up stands and grips (often called “Pop-sockets”), and clips for smart phones, and each separately serves a useful purpose. Pop-up stands and grips for smartphones are well known. An example can be seen in U.S. Pat. No. 8,560,031. The pop-up stand is made from a cone of flexible material and can be pressed flat or extended out, accordion like, to create a conical component that can be used as a grip to hold the smartphone. The extended pop-up stand can also be used as a stand when placing the smartphone on a surface, with the phone placed lengthwise on a flat surface, and set at an angle. While this position is useful for watching videos on a smartphone, there is a need for a device that can be used as a stand to place the phone vertically. Wallet sleeves are also well known and a convenient way to carry identification and credit cards with the phone. These wallet sleeves are relatively flat sleeve openings that can hold a few credit or identification cards, and mount on the back of the phone, or a phone case. The conventional wallet sleeve is typically placed somewhere in the middle of the phone so that when the cards are pushed out of the wallet they are still on the body of the phone, which makes them difficult to retrieve. There is a need, therefore, for a device that attaches the wallet sleeve in a position for easy access to the cards in the sleeve. Phone clips that allow the phone to be attached to a belt, waist band of pants, inside of purse, or other surface, are also well known. There are also wallet sleeves with clips attached, but these are oriented so that the open part of the wallet hangs downward, which means that it is possible for the contents of the wallet sleeve to fall out when the phone is clipped to the user's belt. There is a need, therefore, for a clip that is attached in a manner to prevent the contents of the wallet sleeve from falling out.

There are pop-sockets that are attached to the wallet sleeve, but they are generally attached in the middle of the wallet sleeve, which increases the overall thickness of the phone and attached components. There are also clips attached to the wallet sleeve. But there are no devices that combine the features of the wallet sleeve, the clip, and the pop-up stand. There is a need, therefore for a combined wallet sleeve with an integrated pop-up stand, and an integrated clip.

Most people also carry keys for their car, office, and home. These are typically carried on a key ring or key fob, and carried in the purse or pocket. It is not uncommon for a person to misplace their keys. It is also not uncommon to misplace a phone. There is a need, therefore for a device, or combination of devices, that conveniently attach keys to a smart phone, and also for securing the keys and smart phone, with wallet sleeve, in the same spot in the home and office.

### SUMMARY OF THE INVENTION

This invention consists of a wallet sleeve that will be attached to a smartphone. The wallet sleeve includes an integrated pop-up stand that is positioned above and at the base of the wallet sleeve to reduce the overall thickness of the combined device. The wallet sleeve also includes an attached clip to allow the wallet sleeve, and attached smartphone, to be attached to a belt or other article of clothing. The clip is attached at the bottom of, and flush with the opening of, the wallet sleeve, and the wallet sleeve is attached to the smartphone so that the opening is flush with the bottom of the smartphone. In this orientation the combined wallet sleeve, clip, and phone bottom form a flat surface that can be used as a vertical stand to hold the phone in the upright position.

The wallet sleeve also includes an attached clip, which is a typical belt clip. It is attached at the bottom of the wallet sleeve, adjacent to the opening, so that when the wallet sleeve is attached to clothing the opening for inserting the credit cards will be at the top of the assembly to prevent cards from falling out of the wallet sleeve. This orientation for the clip, wallet sleeve, and smartphone means that when attached the smartphone will be hanging with the top down, but when the user wants to view the phone they merely need to rotate the phone on the clip and it will be oriented so that they are looking down at the face of the phone in the proper orientation.

There is an attachment magnet attached at the top of the pop-up stand. The invention also includes an attachment disc which has one side having an adhesive attachment to attach the attachment disc to a flat surface, and a magnetic side. This allows the attachment disk to be used as an attachment point for the other components of the device. The invention further includes a magnetic key fob, which has a conventional loop for attaching keys, and a flat disk end with two circular magnets on either surface. The magnets allow the key fob to be attached to any metal surface, or the wallet sleeve pop-up stand, and also allow the key fob to be attached to the attachment disk. The pop-up stand also includes an attachment magnet located on the top of the pop-up stand that allows the smartphone to be attached to any metal or magnetic surface. The pop-up stand magnetic disk allows the smart phone to be attached to the magnetic key fob so that a user will have both keys, phone, and wallet, stored together. This configuration also allows the user to attach keys, wallet, and phone to the attachment disk so that all can be secured together.

### BRIEF DESCRIPTION OF THE DRAWINGS

FIG. 1 is a perspective view of the three main components of the invention, the combo wallet sleeve, the magnetic key fob, and the attachment disk.

FIG. 2 is a perspective view of the combo-wallet sleeve with the pop-up stand in the retracted position.

FIG. 3 is a perspective view of the combo-wallet sleeve with the pop-up stand in the extended position.

FIG. 4 is a view of the back of the combo wallet sleeve.

FIG. 5 is an exploded view of the components on the back of the wallet sleeve.

FIG. 6 is a bottom view of the combo wallet sleeve showing the bottom opening and the interior opening.

FIG. 7 is a top view of the combo wallet showing the top and the pop-up stand.

FIG. 8 is an exploded side view of the combo wallet sleeve showing the disassembled components of the pop-up stand.

FIG. 9 is a side view of the combo wallet sleeve showing the clip and the pop-up stand in the retracted position.

FIG. 10 is side view of the combo wallet sleeve showing the clip and the pop-up stand in the extended position.

FIG. 11 is a perspective view of the magnetic key fob.

FIG. 12 is a front view of the magnetic key fob, with the rear view a mirror image thereof.

FIG. 13 is a side view of the magnetic key fob, with both sides identical.

FIG. 14 is a perspective view of the magnetic key fob with keys attached and the fob magnetically attached to a flat metal surface.

FIG. 15 is a perspective view of the attachment disk.

FIG. 16 is an exploded view of the attachment disk.

FIG. 17 is a perspective view of the combo wallet sleeve attached to a smartphone, and with the pop-up stand retracted.

FIG. 18 is a perspective view of the combo wallet sleeve attached to a smartphone, and with the pop-up stand extended.

FIG. 19 is side view of the combo wallet sleeve in position to be attached to the magnetic key fob, which is in position to be attached to the attachment disk.

FIG. 20 is side view of the combo wallet sleeve attached to the magnetic key fob, which is attached to the attachment disk.

### DETAILED DESCRIPTION OF THE INVENTION

Detailed embodiments of the present invention are disclosed herein. It is to be understood that the disclosed embodiments are merely exemplary of the invention, and that there may be a variety of other alternate embodiments. The figures are not necessarily to scale, and some features may be exaggerated or minimized to show details of particular components. Therefore, specified structural and functional details disclosed herein are not to be interpreted as limiting, but merely as a basis for teaching one skilled in the art to employ the varying embodiments of the present invention.

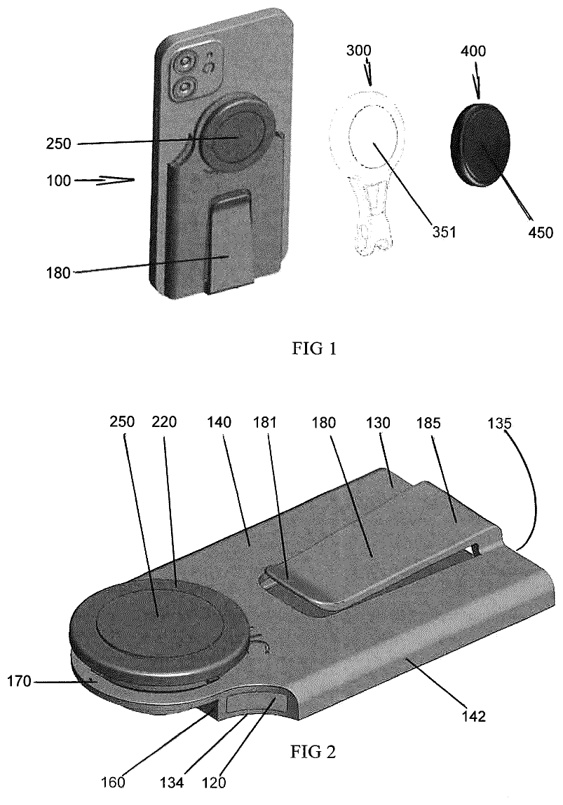

FIG. 1 shows the three main components of the present invention side by side, which are the smartphone wallet sleeve 100, the magnetic key fob 300, and attachment disc 400. The specific elements of each component will be described more fully below. In the present invention, the attachment disc 400 has an adhesive attachment 460 on one side, which allows the attachment disc 400 to be attached to a wall or desk or mirror, or other flat surface preferred by the user, and a disc magnet 450 on the other side. The magnetic key fob 300 includes a key ring 310 for attaching keys, and a disc end 320 with a front magnet 351 and a rear magnet 352, which allows the key fob 300 to be magnetically attached to the attachment disc 400. The smartphone wallet sleeve 100 includes an integrated pop-up stand 200 which includes an attachment magnet 250 at the end of the pop-up stand 200, which allows the smartphone wallet sleeve 100 to attach magnetically to either the mounted attachment disc 400 to attach the smartphone to a wall or other surface, or to the key fob 300, to attach the keys to the wallet sleeve 100 and smart phone. It is also possible for the key fob 300 to attach to the attachment disc 400 and then the smartphone wallet sleeve 100 to attach to the key fob 300. This allows the user to safely and securely keep their phone, wallet and keys all in one simple and convenient place. In one embodiment there will be multiple attachment discs 400 sold or used with the product, so that the user can use one attachment disc 400 at work to hold wallet, keys, and phone, and another to hold wallet, keys, and phone at home, and even in multiple locations in the home, such as on a bathroom mirror and on a wall in a home gym.

The wallet sleeve 100 consists of a wallet housing 110 which has an internal opening 120, a clip 180 attached to the front 140 of the housing 110, a pop-up stand 200 with an integrated attachment magnet 250, which is attached at the top 160 of the housing 110. The components of the wallet sleeve 100 are shown in detail in FIGS. 2-12, and described in detail below. The wallet sleeve 100 attaches to a standard smartphone as shown in FIGS. 8 & 9. The internal opening 120 is sized to accommodate credit cards and identification cards. The interior opening 120 has a inside front wall 114, an inside back wall 115, an inside left wall 132, an inside right wall 131, an inside top wall 136, and an open bottom 135. These correspond to the same components of the housing, specifically the front 140, the back 150, the top 160, the right side 141 and the left side 142. The hosing 110 is made from molded plastic, most preferably conventional thermoplastic, but other known moldable plastics can be used. Some of the features of the housing are functional for the present invention, while others are determined by the requirements of molded plastic.

Both credit cards and government issued ID cards are 3.375 inches (85.6 millimeters) long by 2.125 inches (53.98 millimeters) wide. This is a standard set by the International Organization for Standardization (ISO) and the 162 member nations in the ISO comply by this standard. The thickness of these cards is not set by the convention and so can vary, but not significantly, and are typically approximately 0.0625 inches (1.5 mm) thick. The wallet sleeve internal opening 120 is just a hair's breadth wider than 53.98 mm wide, and is in fact 54 mm wide, which prevents the cards from catching or wedging in the interior opening 120, and prevents foreign objects, like paper from preventing a card from being inserted or removed from the opening.

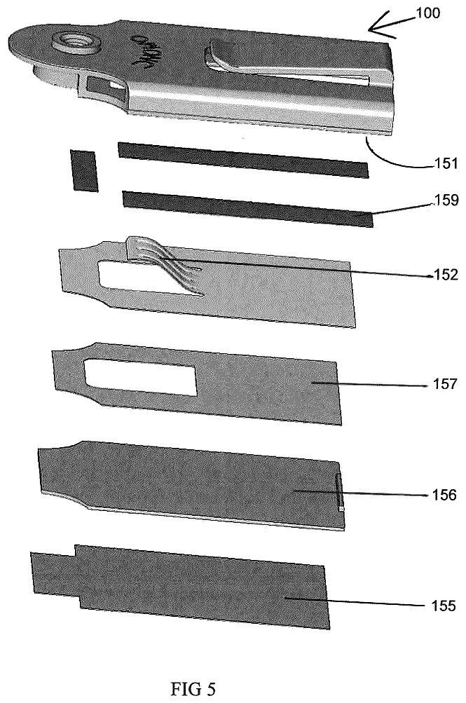

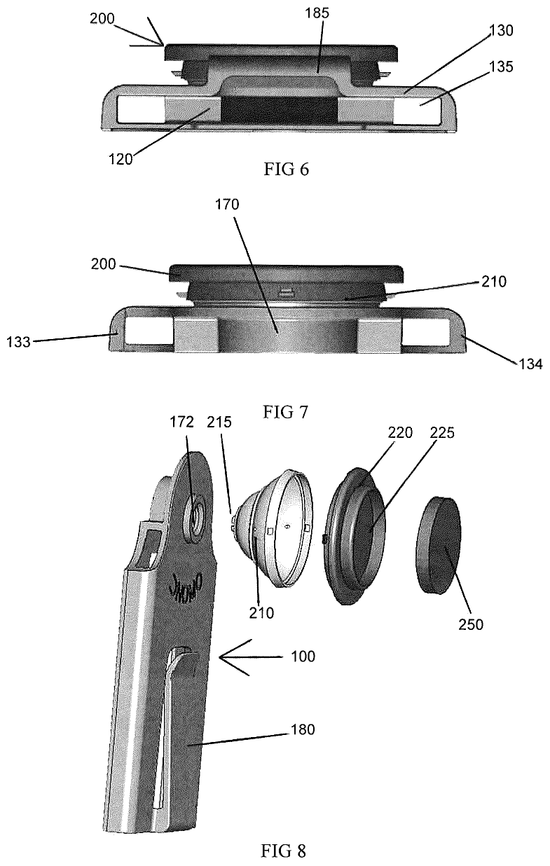

There is a bottom opening 135 of the wallet sleeve 100 at the bottom 130 of the housing 110, best seen in FIG. 6, the bottom view of the combo wallet sleeve. The bottom opening is 54 mm wide, and one-half inch, or 12.7 mm high, and which opens into the internal opening 120. The bottom opening 135 and internal opening 120 have the same dimensions so that the cards do not catch or bind at the bottom opening 135. The height of the bottom opening 135 and internal opening 120 is thick enough to hold between 8 and 9 cards of standard thickness. The fact that the two openings are just slightly wider than a card means that cards will not generally slip out unless pushed. However, obviously, this does not fully secure the cards into the slip wallet 100, particularly when there are only one or two cards.

To prevent the cards from inadvertently falling out of the slip wallet opening 120, there is an internal retaining spring 152 to hold cards in place. The retraining spring 152 is best seen in FIG. 4, the back view of the combo wallet sleeve 100. The retaining spring 152 is a small piece of flexible material, preferably thin spring steel, or possibly flexible plastic. It is approximately one inch wide and two inches long, and has a shallow “S” curve. It is attached to the inside back wall 115 of the opening 120 and curves down to just lightly touch the inside bottom or inside front 114 of the opening 120, so that as cards are slide into the internal opening 120 they push against the retaining spring 152, and the retaining spring 152 pushes them down to temporarily secure them against the inside back 115 of the opening 120. In one embodiment the retaining spring 152 is made from a thin flexible piece of spring steel, which can be attached to the inside back 115 by gluing, or there can be small plastic slips molded on either side of the retaining spring 152 to hold it in place. In an alternate embodiment the retaining spring 152 can be molded from the same molded plastic as the housing 110, and in fact can be a portion of back 150 of the housing 110, and molded in that configuration.

In one embodiment, the retention spring 152 is molded with the housing 110 and in that embodiment there is an opening 153 in the back 150, which is configured into the spring material for the retaining spring 152. In a second embodiment, the retention spring is part of a larger sheet of this spring steel, as shown in FIG. 5. In this embodiment the sheet for the retention spring 152 is attached to the back 150 of the housing 110, by means of adhesive strips 159, with the retaining spring 152 into the opening 153. There is a protective cover 156 that is attached to the spring material by means of an adhesive 157. Finally a two sided adhesive strip 155 is attached to the back of the protective cover 156, and the adhesive strip 155 is used to attach the wallet sleeve 100 to a smartphone.

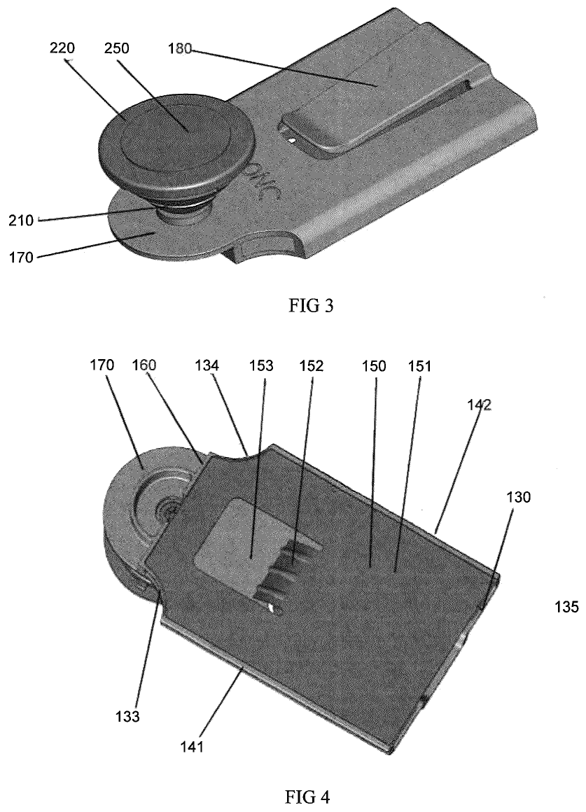

The inside top end 136 of the wallet sleeve 100, the interior opening 120 is closed off to prevent cards from simply sliding through and out, but there are two small quarter-circle finger indents, a left finger indent 134 and a right finger indent 133, as seen in FIGS. 2, 3 & 4. The left and right finger indents 133 and 134 are cut into the left and right sides 141 and 142, and to into the interior opening 120. Cards are inserted into the interior opening 120 and slid forward until they hit the inside top end 136, and when they are fully inserted the top two corners of the cards are visible in the finger indents 133 and 134. When the user wishes to remove the cards they can be pushed out of the internal opening 120 by pushing on the exposed corners of the cards at the finger indents 133 and 134. (It should be noted that the left and right openings are the orientation on the wallet sleeve and not related to the fingers used to push the cards out of the sleeve.) This will push the bottom of the cards out of the bottom 135 of the internal opening 120 so that the user can grab the cards and remove them from the wallet sleeve 100.

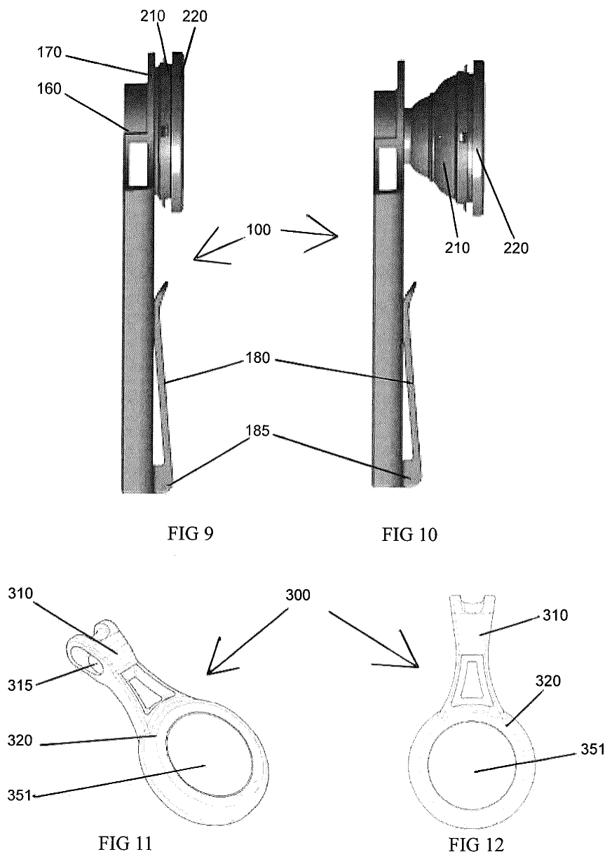

At the top end 160 of the wallet sleeve 100 there is an attachment semi-circle 170 where the pop-up stand 200 is attached, as seen in FIGS. 2, 3, & 4. The attachment semi-circle is a relatively flat piece of molded plastic created in the same mold as the housing 110, and molded to and in the same plane as the front 140 of the housing 110. There is an attachment hole 172 in the center of the attachment semi-circle 170, which mates with the attachment prongs 215 of the pop-up stand. The main components of the pop-up stand 200 are show in the exploded view of FIG. 8. The primary component of the pop-up stand 200 is the flexible accordion cone 210, which can be contracted to a nearly flat configuration, or extended—or popped-up—to create the stand or holder. The pop-up stand 200 is shown in the retracted position in FIG. 8, and in the extended position in FIG. 9. The accordion cone 210 consists of rigid and flexible concentric walls that fold into each other, rather than on each other as in a standard accordion. In the preferred embodiment the accordion cone 210 has three rigid circumferential walls and two flexible walls, with the diameter (and hence circumference) of the walls increasing as they move away from the bottom, where the attachment prongs 215 are attached. At the top of the accordion cone 210 there is a circular magnet holder 220, which attaches to the top of the accordion cone 210 to secure the magnet 250 to the pop-up stand, and has a central magnet disk holder 225 which is sized and configured to hold the magnet 250. The magnet 250 is inserted into the central magnet disk holder 225 and attached. It is possible for the circumference of the magnet disk holder 225 to be essentially the same as the circumference of the magnet 250 so that the magnet 250 can be securely attached into the magnet disk holder 225. It is also possible to secure the magnet 250 into the magnet disk holder 225 for gluing, or through the use of two sided tape or adhesive material. The attachment magnet 250 can be made of high strength magnetic material such as neodymium or other similar magnetic materials. The high strength magnet of this type has a sufficient attachment or adhesive force to allow the smartphone to be attached, and have the smartphone not move once attached. The pull-strength, however, is only a few pounds, so the user can easily pull the smartphone off of the surface. The same material is used in all the attachment magnets 250, 351, 352, and 450.

On the back 150 of the wallet sleeve 100, shown in FIG. 4, there is the phone attachment surface 151. In some embodiments the wallet sleeve 100 is attached to the back of the phone, or the phone case, by an adhesive strip 155 which is attached to the back 150 of the wallet sleeve 100. In other embodiments that wallet sleeve is attached to the phone by a plastic phone protective housing that is snapped onto the phone.

On the front 140 of the wallet sleeve there is a clip 180, which is made of the same molded plastic material as the housing 110 of the wallet sleeve 100. In the most preferred embodiment the clip 180 is formed in the same mold as the housing 110. The clip 180, show in in FIGS. 2, 3, 9 & 10, is a conventional and well known clip, is used to attach the wallet sleeve 100, and the smartphone to which it is attached, to a belt or waist band of pants, or other clothing, so that a user can secure their phone to their clothing for ease of access and use. The clip 180 can also be used to secure the phone and wallet sleeve 100 to a purse or pocket on or inside a purse, a satchel, backpack, or any other common carrying item. In the preferred embodiment, the clip 180 is molded plastic and molded in the same mold as the rest of the housing 110, and the clip attachment 185 is mounted to and attached to the bottom 130 of the housing 110. As best seen in the side views of FIG. 8 & FIG. 9, the bottom of the clip attachment 185, and the bottom 130 of the wallet sleeve 100 form a flat surface of approximately one inch wide, which creates a flat bottom stand surface for the phone. When the phone is attached to the wallet sleeve 100 the bottom flat surface is more than an inch wide, and can easily be used as a flat stand that allows the phone to be positioned on a flat surface in the upright position.

In the preferred embodiment the belt clip 180 is molded from the same material, and as part of the wallet sleeve 100 so that they are an integrated unit. The clip 180 is attached to the bottom 130 of the wallet sleeve 100 so that when the phone is attached to the wallet sleeve 100, the phone will be oriented downward when attached to a belt or waist band of clothing. In this orientation, the user can flip the phone outward, and be looking down at a properly oriented phone screen. This allows the user to easily check a text message or a phone number when the phone is attached to the belt or waist band of clothing. As can be seen in the side view of FIG. 9, when the pop-up stand 200 is in the retracted position, the top of the pop-up stand is only slightly raised above the front 140 of the housing 110, and only slightly extended from the clip 180, to create a relatively flat side profile for the combo wallet sleeve 100. This prevents the pop-up stand 200 from potentially conflicting with clothing when attached by the clip 180.

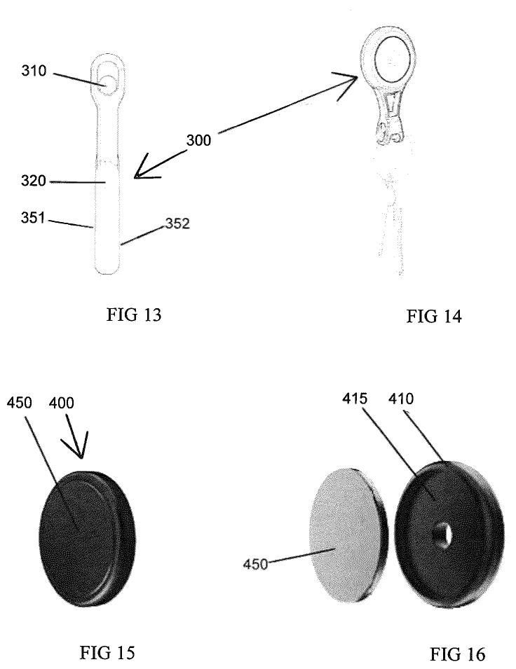

FIG. 11 is a perspective view of the magnetic key fob 300 which is a substantially tear-drop shape with a key ring end 310 at the top and a larger circular disc end 320 at the bottom. FIG. 12 is a front view, and FIG. 13 is a side view of the key fob 300. In the preferred embodiment the magnetic key fob 300 is made of conventional molded plastic, which can be easily molded to produce all of the desired features. The circular disc end 320 is approximately 1½ inches in diameter, and approximately one-half inch thick. In the center of the circular disc end 320, on both the front and back, there is a magnet recess, with the front recess 321 on the front and the back recess 322 on the back. Each recess 321 and 322 is one inch (1″) in circumference and one-eight inch (⅛″) deep, and sized to hold a circular magnet, the front magnet 351 and the back magnet 352. These magnets are the same size and made from the same material as the magnets on the attachment disc 400, and the smart phone wallet 100 pop-up stand 200, and are sized and configured to magnetically attach to those magnets. The recesses 321 and 322 will be precisely sized so that the magnets 351 and 352 will snap into the recess and be securely attached. It is also possible, and within the conception of the invention to securely attach the magnets within the recesses by means of glue or double-sided adhesive tape.

The key fob 300 tapers away from the circular disc end 320 to the key ring end 310, which is approximately three-quarters of an inch (¾″) wide and roughly one-half inch thick. The entire key fob 300 is approximately two and one-half inches long (2½″), with a slight waist between the circular disc end 320 and the key ring end 310. At the key ring end 310 there is an attachment loop 315 which is a hole running through the key ring end 310. The attachment loop 315 is perpendicular to the circular disc end 320 so that when a key ring is attached within the attachment loop 315 the key ring itself is in roughly the same plane as the circular disc end 320, as can be seen in FIG. 14, which shows the fob 300 attached to a magnetic surface. This allows the key ring to move within the attachment loop 315, and so that, when the key fob 300 is attached to either (or both) of the attachment disc 400 or the wallet sleeve 100 on the phone, they key ring can move so that the keys on the ring do not conflict with the phone or the wall. The magnetic key fob 300 can be used in conjunction with the attachment disc 400, and with the smart phone wallet sleeve 100, as described below. But it can also be used independently to attach keys to any magnetic surface.

FIG. 15 is a perspective view of the attachment disc 400 and FIG. 16 is an exploded view of the attachment disc 400 to show all of the elements. As is best seen in the exploded view of FIG. 16, the attachment disc 400 consists of a disc holder 401, which in the preferred embodiment is a disc of molded plastic with a diameter of roughly one and one-quarter inch. There is a front recess 405 in the disc holder 401 which is a one-inch (1″) diameter recess of approximately one-eight inch (⅛″) depth. There is a circular disk magnet 450 that fits into the recess 405, so the circular magnet 450 is one inch diameter and one-eight of an inch thick (1″ by ⅛″). The back of the disc holder 401 is flat, and there is a circular piece of attachment adhesive 430 attached. This allows the attachment disc 400 to be attached to any flat surface. The circular magnet 450 is conventionally attached into the circular recess 405, which in the preferred embodiment is by gluing. Note that in FIG. 15 there is a small circular hole in the middle of the attachment disc 400. This is for the molding process, and has no functional component and is not par to the invention. In one embodiment of the invention designating stickers can be attached to the circular magnet 450 in the middle of attachment disc 400, to personalize the attachment disc 400, and to identify it in a situation where multiple people, such as in a family house, may have attachment discs 400.

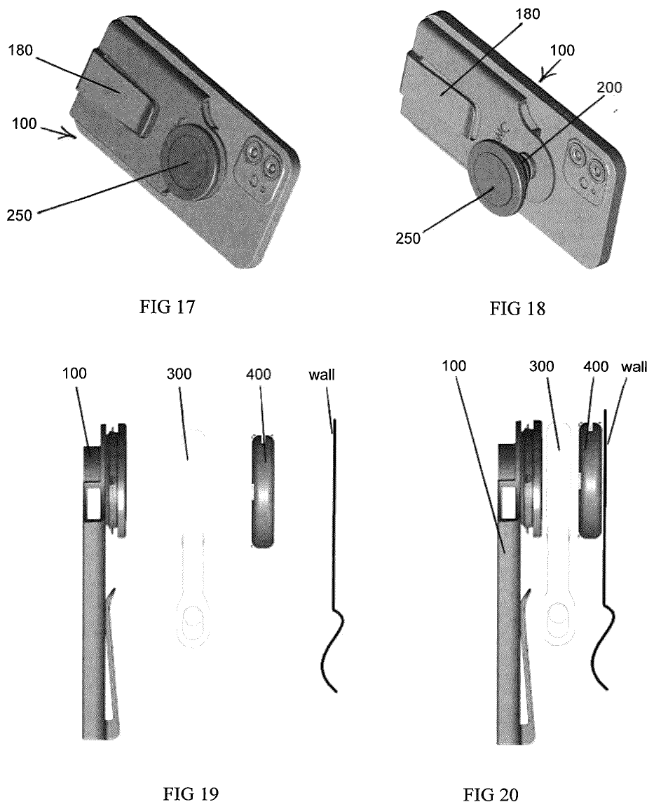

In use, the user attaches the wallet sleeve 100 to the phone by removing the covering of the adhesive strip 155 on the back 150 of the wallet sleeve 100 and attaching the wallet sleeve 100 to the back of the phone so that the bottom 130 of the wallet sleeve 100 is even with the bottom of the phone. FIG. 17 and FIG. 18 show the wallet sleeve 100 attached to a smart phone, with FIG. 17 showing the pop-up stand retraced, and FIG. 18 showing the pop-up stand extended. When the wallet sleeve 100 is attached to the phone in this manner it creates a flat surface comprising the bottom 130 of the wallet sleeve 100, the bottom of the clip attachment 185, and the bottom of the phone, which is a large enough surface to create a flat stand for the phone. This allows the phone to stand upright on a flat surface. Additionally, when the wallet sleeve 100 is attached to the phone in this manner, the clip 180 is situated so that when the smartphone is attached to a belt or waist band the phone is hanging down with the face of the phone out. When the user wants to view the phone, they can simply rotate the top of the phone up, and the face of the phone will be oriented so that the user can see the information presented on the screen.

Once the wallet sleeve 100 is attached to the phone the user can insert cards into the interior opening 120. Even a single card will be secured in the interior opening because the internal retention spring 152 will hold the card in place. As more cards are inserted the retention spring 152 will move but will allow more cards, in the preferred embodiment up to eight cards to be inserted into the internal opening 120. The end corners of the cards will visible at the right finger indent 133 and the left finger indent 134, and the user can push the cards out of the bottom opening 135 by pushing against the card corners at the left and right finger indents 133 and 134, which will expose enough of the cards at the bottom opening 135 to allow them to be removed.

The clip 180 is attached to the front 140 of the wallet sleeve 100, and is attached at the bottom 130 so that when the wallet sleeve 100 is attached to a belt or waist band of pants or a skirt, the phone is oriented downward. This places the bottom opening 135 at the top of the downwardly hanging phone and wallet sleeve 100 so that the cards in the wallet sleeve 100 cannot fall out. Once the user attached the phone, by means of the clip 180, to the waist band, the phone will be oriented downward, but when the user swings the phone up and out, the user will be looking down at the face of the phone in the correct orientation. The clip attachment 185 is oriented at, and molded to the bottom 130 of the housing 110, and the bottom of the clip attachment 185 and the bottom 130 of the housing 110 form a flat surface of roughly ¾″, which means that when the phone is attached there is a flat surface of about one inch in width and about three inches long. This is a sufficient flat surface to create a flat bottom stand for the phone, which will allow the user to set the phone down in the upright position to view videos on the phone, as a convenient stand to use the phone camera for facetime of video chats, or to be used as a stand for taking pictures with the phone.

If the user prefers, the user can use the pop-up stand 200 and a convenient grip to hold the phone, or as a stand to set the phone on its side and angled up so that the user can watch videos on the phone, or as a stand form using the phone for facetime of video calls. The pop-up stand 200 can extend out to be used as a grip of stand, and retracts in to present a fairly flush profile. When the pop-up stand 200 is retracted it has a side profile width of just over one-half of an inch, and not much higher than the clip 180. This is flat enough so that it will not conflict with clothing when the phone is attached with the clip 180.

The user can use the attachment adhesive 430 to mount the attachment disc 400 to any convenient flat surface such as a mirror in the bathroom at home, the dashboard in the car, or the side of a desk at the office. It is within the conception of the invention to have multiple attachment disks 400, and attach them in multiple places, such as the office, car dashboard, at home by the front door, in the bathroom, or even a home gym. The user can attach their keys to the magnetic key fob 300 at the attachment loop 315, and can attach the magnetic key fob 300 to the attachment disk 400 by means of either one of the front magnet 351 or rear magnet 352, it doesn't matter which since the key fob 300 is a simple and relatively flat component.

In the preferred embodiment all three components can be attached to keep them all in a safe and known place. FIG. 19 shows the attachment disk 400 in position to be attached to a flat surface, the key fob 300 in position to be attached to the attachment disk 400, and the magnet 250 of the wallet sleeve 100 in position to be attached to the key fob 300. FIG. 20 shows the three components attached. This will keep the keys secure and in a handy, and known, place. The pop-up magnet 250 can be used to attach the magnetic key fob 300 to the smartphone so that the users keys, slip wallet 100, and smart phone are all in the same place. The combination can also be attached to the pre-positioned attachment disk 400, which allows the user to store the phone, wallet, and keys in the same place, thus reducing the common experience of forgetting where you put your phone, keys or wallet. With this system they will always be in the same place and always together.

The present invention is well adapted to carry out the objectives and attain both the ends and the advantages mentioned, as well as other benefits inherent therein. While the present invention has been depicted, described, and is defined by reference to particular embodiments of the invention, such reference does not imply a limitation to the invention, and no such limitation is to be inferred. The depicted and described embodiments of the invention are exemplary only, and are not exhaustive of the scope of the invention. Consequently, the present invention is intended to be limited only be the spirit and scope of the claims, giving full cognizance to equivalents in all respects.

I claim:

1. A device for securing a wallet, cell phone, and keys, said device comprising:
an attachment disc having one adhesive side and one magnetic side, wherein said attachment disc is flat and has a first side with adhesive to attach to a flat surface, and a second side with a flat disc shaped attachment magnet;
a magnetic key fob with a ring end and a disk end, wherein a key loop is integrated at the ring end, and wherein said key loop is configured to attach to a conventional key ring, and wherein said disk end consists of a flat front surface having a front key fob attachment magnet and a flat rear surface having a rear key fob attachment magnet, and wherein further either of the key fob attachment magnets are sized and configured to magnetically, and temporarily, attach to the disc shaped attachment magnet of the attachment disc;
a smart phone combo wallet having a pop-up stand, wherein said smart phone combo wallet is configured to attach to a standard smart phone, and wherein said smart phone combo wallet has a flat wallet sleeve sized and configured to hold identification and credit cards, and wherein said smart phone combo wallet also has an attachment clip configured to attach the smart phone combo wallet (with or without the phone) to a belt or the waist band of a pair of pants (and configured such that the phone is handing top down so that when lifted up the user can see the phone in the correct orientation), and wherein the wallet sleeve has two finger inlets on the top to push out the cards, and an internal (spring to hold the cards in place,) and wherein the pop-up stand has a magnetic attachment disc on the “top” external part of the pop-up stand,
wherein the magnetic attachment disc of the pop-up stand can attach directly to the disc shaped magnet of the attachment disc to hold the phone on a designated flat surface, can be attached to the magnet of the key fob to secure the keys with the phone, and can be attached to the key fob with the key fob attached to the attachment disc on a designated surface to secure the wallet, cell phone and keys in a specified place.

2. The device of claim 1 wherein said attachment clip is attached so that the bottom of the attachment clip is aligned with the bottom of the wallet sleeve, and wherein further the bottom of the wallet sleeve is aligned with the bottom of the smart phone, such that a platform is created.

3. The device of claim 1 wherein the attachment disks of the key fob allow the key fob to be magnetically attached to any metal surface, to the attachment disk, to the magnet of the pop-up stand, or to both.

4. The device of claim 1 wherein the pop-up stand attachment magnet allows the slip wallet to be magnetically attached to any metal surface.
